(19) United States
(12) Patent Application Publication
(10) Pub. No.: US 2020/0388359 A1
(43) Pub. Date: Dec. 10, 2020

(54) METHODS AND SYSTEM OF BLOCKCHAIN-BASED MEDICAL SERVICE MANAGEMENT

(71) Applicants: Rekha Bhandari, Woodbury, NY (US); Pavana Jain, Redwood City, CA (US)

(72) Inventors: Rekha Bhandari, Woodbury, NY (US); Pavana Jain, Redwood City, CA (US)

(21) Appl. No.: 16/780,585

(22) Filed: Feb. 3, 2020

Related U.S. Application Data
(60) Provisional application No. 62/799,754, filed on Feb. 1, 2019.

(51) Int. Cl. G16H 10/60; G16H 50/20; G16H 20/30; H04L 12/58

(52) U.S. Cl. CPC G16H 10/60; G16H 50/20; H04L 67/10; H04L 51/02; G16H 20/30

(57) ABSTRACT

In one aspect, a computerized method for implementing a patient identification application to access patient information in a blockchain system includes the step of providing a patient identification application. The patient identification application is provided and implemented in a patient-side computing systems and a health-care side computing systems. The method includes the step of securely storing a patient identification information in a blockchain system. The method includes the step of securely storing a patient medical history in the blockchain system. With a health-care side computing system, the method obtains the patient identification information of the patient from the blockchain system.

### CROSS-REFERENCE TO RELATED APPLICATIONS

This application is a continuation in part of U.S. Patent Provisional Application No. 62/799,754, titled METHODS AND SYSTEM OF BLOCKCHAIN-BASED MEDICAL SERVICE MANAGEMENT and filed on 1 Feb. 2019. This application is hereby incorporated by reference in its entirety.

### FIELD OF THE INVENTION

The invention is in the field of blockchain-based cryptography and more specifically to a method, system and apparatus of blockchain-based medical service management.

### DESCRIPTION OF THE RELATED ART

The healthcare industry is faced with deeply fragmented and proprietary information systems at the hospitals, health providers, labs, insurance companies, home care providers, public policy websites, communities, and preventive health data providers. Accordingly, the consumer does not own his/her health data and cannot share it easily, securely, and in real time. Consumers are unaware of impacts of medications, chronic diseases, repeating tests, diagnosis, and treatments. They are also not fully aware of preventive habits that they can help them.

### BRIEF SUMMARY OF THE INVENTION

In one aspect, a computerized method for implementing a patient identification application to access patient information in a blockchain system includes the step of providing a patient identification application. The patient identification application is provided and implemented in a patient-side computing systems and a health-care side computing systems. The method includes the step of securely storing a patient identification information in a blockchain system. The method includes the step of securely storing a patient medical history in the blockchain system. With a health-care side computing system, the method obtains the patient identification information of the patient from the blockchain system.

### BRIEF DESCRIPTION OF THE DRAWINGS

FIG. 1 illustrates an example system for managing medical and other healthcare related services with a blockchain system, according to some embodiments.

FIG. 2 depicts an exemplary computing system that can be configured to perform any one of the processes provided herein.

FIG. 3 is a block diagram of a sample computing environment that can be utilized to implement various embodiments.

FIG. 4 illustrates an example process for managing consumer health records with a blockchain system, according to some embodiments.

FIG. 5 illustrates an example process for implementing a patient identification application to access patient information in a blockchain system, according to some embodiments.

FIG. 6 illustrates an example process for implementing an AI functionality to continuous monitor and advise patients, according to some embodiments.

FIG. 7 illustrates an example process for managing diabetes with a blockchain, according to some embodiments.

FIG. 8 illustrates an example process for monitoring a patient's mental state with an AI functionality.

The Figures described above are a representative set and are not an exhaustive with respect to embodying the invention.

### DESCRIPTION

Disclosed are a system, method, and article of manufacture for blockchain-based medical service management. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment,’ ‘an embodiment,’ ‘one example,’ or similar language means that a particular feature, structure, or characteristic described in connection with the embodiment is included in at least one embodiment of the present invention. Thus, appearances of the phrases ‘in one embodiment,’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

Example definitions for some embodiments are now provided.

Application programming interface (API) can specify how software components of various systems interact with each other.

Blockchain is a growing list of records, called blocks, which are linked using cryptography. Each block contains a cryptographic hash of the previous block, a timestamp, and transaction data (e.g. represented as a Merkle tree root hash).

Cloud computing can involve deploying groups of remote servers and/or software networks that allow centralized data storage and online access to computer services or resources. These groups of remote serves and/or software networks can be a collection of remote computing services.

Cryptocurrency can be a digital asset designed to work as a medium of exchange that uses strong cryptography to secure financial transactions, control the creation of additional units, and verify the transfer of assets.

Decentralized application (Dapp) is an application that is run by many users on a decentralized network with trustless protocols. A Dapp can be used to avoid any single point of failure.

Electronic health record (EHR) is the systematized collection of patient and population electronically-stored health information in a digital format. An EHR can be shared across different health care settings. Records can be shared through network-connected, enterprise-wide information systems or other information networks and exchanges. EHRs can include a range of data, including, inter alia: demographics, medical history, medication and allergies, immunization status, laboratory test results, radiology images, vital signs, personal statistics like age and weight, and billing information.

Ethereum is an open-source, public, blockchain-based distributed computing platform and operating system featuring smart contract (scripting) functionality.

Machine learning is a type of artificial intelligence (AI) that provides computers with the ability to learn without being explicitly programmed. Machine learning focuses on the development of computer programs that can teach themselves to grow and change when exposed to new data.

Example Blockchain System for Managing Patient Healthcare

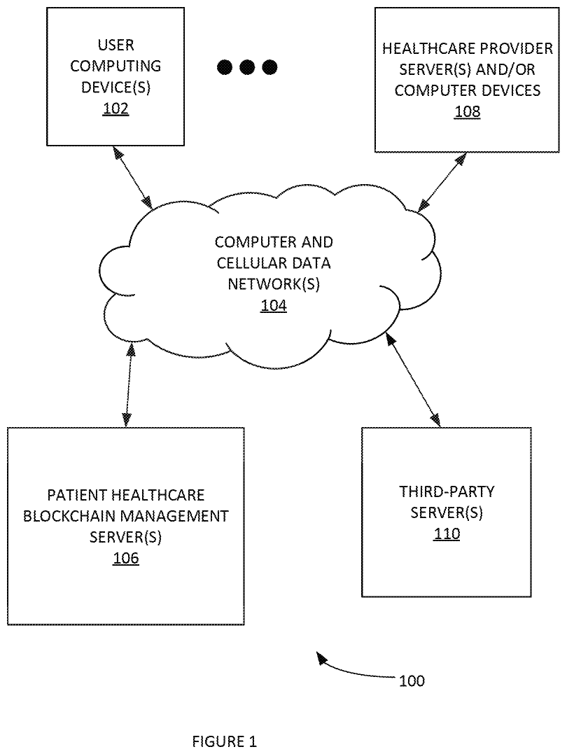

FIG. 1 illustrates an example system for managing medical and other healthcare related services with a blockchain system, according to some embodiments. System 100 can include user computing device(s) 102. Patients/consumers can use to access patient healthcare blockchain management services. In some examples, user computing device(s) 102 can include a patient healthcare blockchain management application. Patients can upload patient information, healthcare history, daily exercise information, daily nutritional information, etc. via the patient healthcare blockchain management application. Patient healthcare blockchain management application can connect the patient with all the patient's past and current health care providers and institutions using a blockchain technology (provided by patient healthcare management server(s) 106). Patient healthcare blockchain management server 106 use the patient healthcare blockchain management application to provide information to the consumers directly, securely and transparently using distributed decentralized applications on their mobile devices in real time.

System 100 can include healthcare provider a healthcare provider version of the healthcare blockchain management application. Healthcare providers can monitor patient behavior, view patient medical history, send questions to patients, etc.

Patient healthcare management server(s) 106 can provide and manage a patient healthcare information system that utilizes a blockchain system. Data obtained by system can be stored in a blockchain system. For example, the blockchain system can provide and manage a distributed ledger managed by a peer-to-peer network collectively adhering to a protocol for inter-node communication and validating new blocks. This distributed network and ledger can include interoperability aspects. Once recorded, the data in the blockchain system in any given block cannot be altered retroactively without alteration of all subsequent blocks, which requires consensus of the network majority. Patient healthcare management server(s) 106 can manage the patient healthcare blockchain application. It is noted that a patient can be a node in the blockchain needs to be introduced. The patients can actively provide updates to timestamped blocks of information. It is noted that there is the ability to scan and share aspect where portions of blockchain or entire blockchain being shared with one or more health provider is a key feature of our offering and is highly patentable.

Patients and healthcare providers interact via the patient healthcare management server(s) 106 in order to make collaborative, informed decisions, diagnose accurately, eliminate redundant tests, and ensure safe transition of care and better health outcome. These interactions between the patient and the provider, enable greater and proactive engagement, resulting in lowered costs by corresponding early intervention and remedy. By lowering the cost for each patient, our solutions drastically reduce the overall health care cost for care health care system while promoting preventive health maintenance habits.

System 100 can include various computer and/or cellular data networks 104. Computer and/or cellular data networks 104 can include the Internet, cellular data networks, local area networks, enterprise networks, etc. Networks 104 can be used to communicate messages and/or other information from the various entities of system 100.

System 100 can include a patient healthcare blockchain management server 106. The patient healthcare blockchain management server 106 can include a decentralized application infrastructure for creating blockchain application. The patient healthcare blockchain management server 106 can support cryptocurrency allocation, redemption and payment options. The patient healthcare blockchain management server 106 can facilitates transaction processing with enhanced data security, auditability, and transparency across all its platform tiers thereby improving trust amongst anonymous participants. The patient healthcare blockchain management server 106 can incorporate digital identity to authenticate users, performs transaction verification, its provenance, and record asset and value exchange on a distributed ledger. This can be shared across peer-to-peer network with self-enforcing smart contracts eliminating the technical dependency common with a single ledger system. Through robust decentralized consensus protocol and real-time execution, the distributed ledger technology is able to achieve faster throughputs. This can be used to automate governance rules for autonomous processing of transactions involving multiple party blockchain agreements. Open APIs and SDKs can be provided such that developers would find practical and useful for cross platform application development and interoperability with external systems. The patient healthcare blockchain management server 106 can be used to develop decentralized application, deploy and test, and rollout solutions on blockchain network, and in fact are the three critical activities that organizations must perform to target opportunities with blockchains.

More specifically, patient healthcare blockchain management server 106 can provide an informed and engaged consumer with a proactive care delivery system. The patient healthcare blockchain management server 106 can help improve population health outcomes serving to bend the healthcare cost curve even more. The patient healthcare blockchain management server 106 enables healthcare providers to go beyond conventional medical treatments and appointment making. By empowering consumers with personalized health information and delivered to their mobile devices, consumers can access it from anywhere, anytime—health becomes their priority. With positive incentives through digital tokens, taking care of oneself becomes a rewarding experience, and this helps in cultivating preventive health habits with greater impact for all.

Patient healthcare blockchain management server 106 can provide and manage various consumer-centric applications that improve patient health. As an example, such consumer-centric applications are diabetes management, medication schedule reminder management, exercise/physical therapy schedules and instructions, patient health state monitoring. The long-term goal of consumer-centric applications are to help patients improve and maintain good health.

It is noted that patient healthcare management server(s) 106 can implement processes 400, 500, 600, 700 and 800. Patient healthcare management server(s) 106 can implement the AI functionality provided infrastructure. Patient healthcare management server(s) 106 can back up patient data to the patient's medical care provider systems. Patient healthcare management server(s) 106 can manage blockchain-based longitudinal health record(s). Patient healthcare management server(s) 106 can provide and manage chat bots and/or other AI-based personal assistants to interact with patients, medical care providers, exercise coaches, etc. The chat bots and/or other AI-based personal assistants access the patient's blockchain-based longitudinal health record(s).

Patient healthcare management server(s) 106 can provide tokenization of patient health care. Patient healthcare management server(s) 106 can determine various suppliers specific to a patient's condition. Patient healthcare management server(s) 106 can personalize how AI learns from user and applies ML and chat bots to provide health information and day to day management advice. Advice can be tailored for each particular patient. Based on consumer attributes (e.g. height, weight, age, sex, health history, etc.) can make recommendations and then tokenization said recommendations.

Patient healthcare management server(s) 106 can maintain a medical service marketplace. For example, patient healthcare management server(s) 106 can determine that a patient is running out of a specific medication and then contact the patient's pharmacy to fill the medications automatically. If it is determined by an AI review of the patient's longitudinal healthcare records that the patient needs to be seen by a doctor, the patient healthcare management server(s) 106 can send an alert that patient is to schedule the appointment (e.g. in the patient's application) or automatically schedule the appoint and send a notification to the patient. Patient healthcare management server(s) 106 can enable health providers to review a patient longitudinal health profile and suggest additional health management actions (e.g. tests, new meds, med interactions, diet changes, doctor visits, etc.).

Patient healthcare management server(s) 106 can implement ML to implement various AI systems. Accordingly, various machine learning, ranking and/or various optimizations can be utilized by the systems and processes provided herein. Machine learning is a type of artificial intelligence (AI) that provides computers with the ability to learn without being explicitly programmed. Machine learning focuses on the development of computer programs that can teach themselves to grow and change when exposed to new data patterns. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning.

Patient healthcare management server(s) 106 can implement predictive analytics. Predictive analytics can combine a retrospective view of the individual clinical health data, related data from public health agencies, patient-sourced social determinants of health, data from wearables/3^{rd}-party data. This data is associated with and securely directed to the patient identity on the blockchain in a way that keeps the data immutable, ensures privacy via encryption, yet still accessible to those with access-permission. Using this information, a predictive analytics core application implemented by Patient healthcare management server(s) 106, along with a preferred-patient interaction modality, can be programmed with appropriate AI rules to guide engagement, diagnosis and connections to appropriate healthcare provider resources/medications/interventions as needed. The semantics of this predictive analytics and associated conversations are delivered to the patient via various interaction modalities such phone call, interactive messaging, application notifications, increasing patient engagement, becoming proactive and compliant.

Patient healthcare management server(s) 106 can maintain a health marketplace portal. This capability enables marketplace suppliers of healthcare products and services to sell these to a consumer/patient they wish to target. The marketplace can also provide for the participation between consumer patients and the market player (hospital, insurance company, enterprise, pharma company, etc.). Such commercial and incentive-based schemes are implemented on the health marketplace blockchain with a smart contract. An example of such a smart contract may be an award of 1 token after the consumer has completed 10,000 steps/day for 30 days. Another example could be an award of 3 tokens to for sharing of “redacted data” with research organizations (e.g. Pfizer Research in the marketplace). Yet another may be 2 tokens to keep A1c blood sugar measure below 5 for two consecutive months.

Patient healthcare management server(s) 106 can implement an opioid use disorder (OUD) management application. Here, the OUD application uses the capability of the blockchain to uniquely ensure the identity of a person—essentially removing the risk of patient misidentification, a well-known vulnerability in OUD. In addition to this unique functionality, it also serves to improve the efficiency of patient identification. Finally, when the OUD application combines unique patient identification capability with assessment of pain in activities of daily living, opioid abuse likelihood/doctor shopping scores, history of medications, and behavioral health—providers can obtain a dashboard of OUD predictive analytics at the point of care, reducing the opportunity for drug misuse, abuse and diversion.

Patient healthcare management server(s) 106 can manage patient health record portability especially important for referrals to/from providers, or when patients move to a different location, change their healthcare provider or even seek a specialist/2nd opinion. It is noted that there may be various methods to establish a serving relationship between the patient and the provider, one embodiment may accomplish this via a specific “access and use” smart contract on the serving blockchain. For this scenario, a block-chain supported smart contract can be used by a provider to obtain access to the latest, current personalized health record with any updates posted back on to the patients' personal record on the blockchain. Since a patient can access this record, they can grant “access and use” to another provider. In this way, a universal digital health record portability can be provided that maintains immutability, integrity and the longitudinal view of a patient record. This may be extended to complementary non-traditional care providers. Examples of such alternative health providers are rehabilitation facilities, yoga, massage, mid-wives, personal coaches, etc. In one embodiment of the blockchain, this can be implemented as a private/permission blockchain (e.g., just serving Medicare patients nationwide). The private/permission blockchain does not preclude the use of a public blockchain, and can increase the membership/reach/access to both patient and provider entities.

Patient healthcare management server(s) 106 can manage patient referential matching. Patient healthcare management server(s) 106 can enable the personalized her on the blockchain to eliminate or reduce misidentification and duplicate medical records at the point of care. It is noted that an existing medical record may contain a patient's old address and maiden name, whereas a more recent but duplicate record contains that same patient's current address and married name. By using the personalized EHR as the gold standard (and not error-prone deterministic or probabilistic models), medical records can be properly linked, medical/medication errors can be avoided, denied claims can be dramatically reduced. For a hospital, payer, clinic or transition care facility, having a full system of such “golden” records results in enhanced patient engagement, dramatic improvements across quality of care, dependable predictive analytics, accurate population health data, virtually error-free stratification for patients-at-risk—all resulting in proper value-based care and reporting. Finally, patient satisfaction goes up with them having the knowledge that their medical record is comprehensive but most importantly, also fully portable.

Additional Example Computer Architecture and Systems

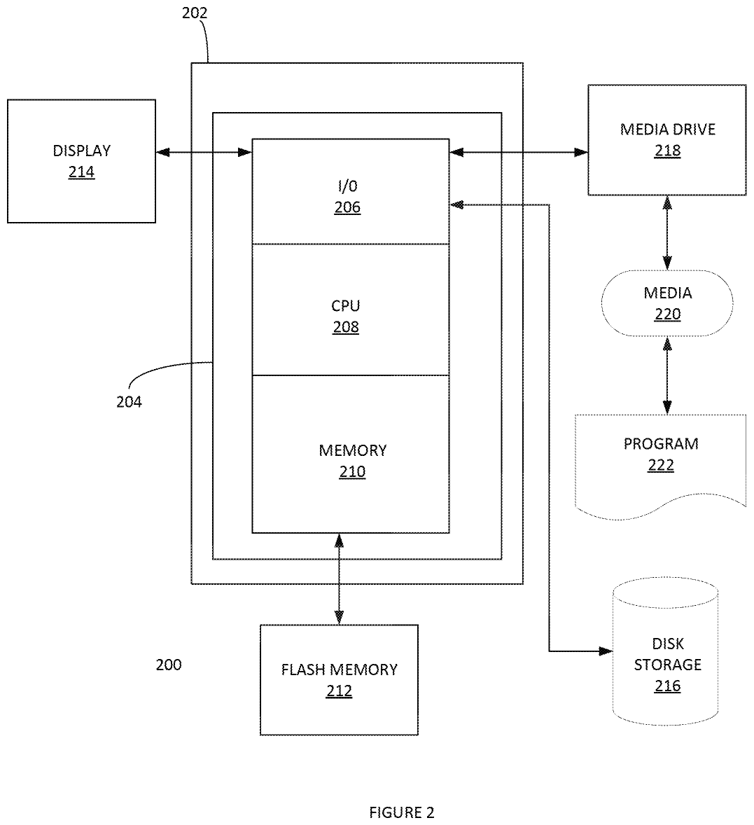

FIG. 2 depicts an exemplary computing system 200 that can be configured to perform any one of the processes provided herein. In this context, computing system 200 may include, for example, a processor, memory, storage, and I/O devices (e.g., monitor, keyboard, disk drive, Internet connection, etc.). However, computing system 200 may include circuitry or other specialized hardware for carrying out some or all aspects of the processes. In some operational settings, computing system 200 may be configured as a system that includes one or more units, each of which is configured to carry out some aspects of the processes either in software, hardware, or some combination thereof.

FIG. 2 depicts computing system 200 with a number of components that may be used to perform any of the processes described herein. The main system 202 includes a motherboard 204 having an I/O section 206, one or more central processing units (CPU) 208, and a memory section 210, which may have a flash memory card 212 related to it. The 1/O section 206 can be connected to a display 214, a keyboard and/or other user input (not shown), a disk storage unit 216, and a media drive unit 218. The media drive unit 218 can read/write a computer-readable medium 220, which can contain programs 222 and/or data. Computing system 200 can include a web browser. Moreover, it is noted that computing system 200 can be configured to include additional systems in order to fulfill various functionalities. Computing system 200 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

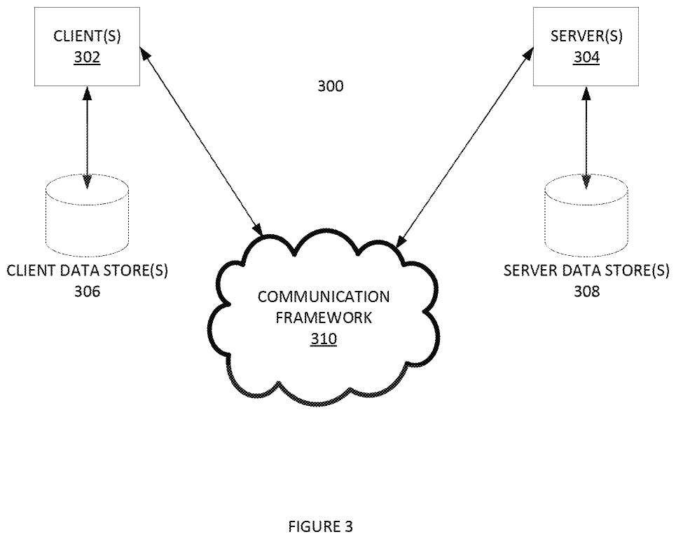

FIG. 3 is a block diagram of a sample computing environment 300 that can be utilized to implement various embodiments. The system 300 further illustrates a system that includes one or more client(s) 302. The client(s) 302 can be hardware and/or software (e.g., threads, processes, computing devices). The system 300 also includes one or more server(s) 304. The server(s) 304 can also be hardware and/or software (e.g., threads, processes, computing devices). One possible communication between a client 302 and a server 304 may be in the form of a data packet adapted to be transmitted between two or more computer processes. The system 300 includes a communication framework 310 that can be employed to facilitate communications between the client(s) 302 and the server(s) 304. The client(s) 302 are connected to one or more client data store(s) 306 that can be employed to store information local to the client(s) 302. Similarly, the server(s) 304 are connected to one or more server data store(s) 308 that can be employed to store information local to the server(s) 304. In some embodiments, system 300 can instead be a collection of remote computing services constituting a cloud-computing platform.

Example Processes

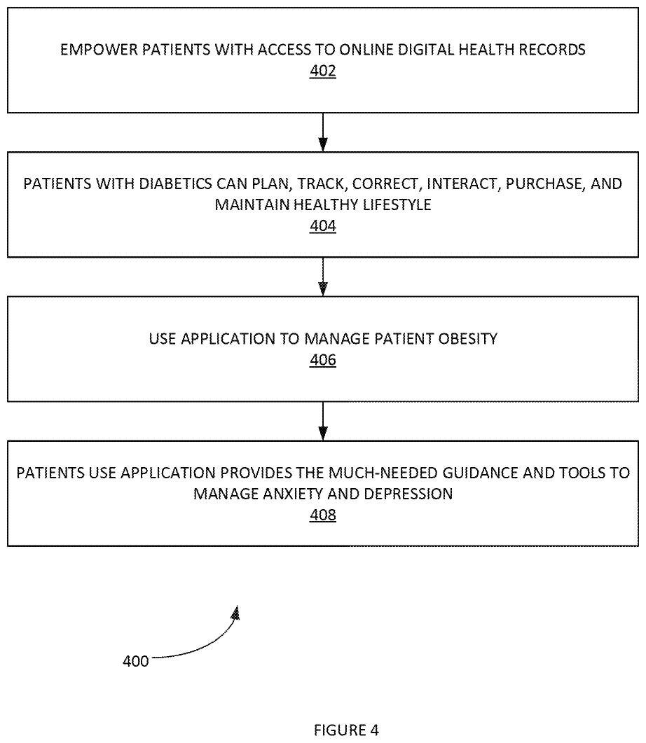

FIG. 4 illustrates an example process 400 for managing consumer health records with a blockchain system, according to some embodiments. Process 400 can empower consumers/patients to access, view and update their own health records which is updated asynchronously in real-time from a variety of sources such as in-patient electronic health records (EHR), out of network provider EHR while they were traveling, wearable devices, etc. Consumers are provided the capability to access and share their health records. For example, a consumer can share his/her health records (or a relevant portion thereof) with a medical provider, insurance company, employer, educational institution, etc. with the assurance that the record obtained from the blockchain is the most current record.

More specifically, in step 402, process 400 can empower patients with access to online digital health records. In step 404, patients with diabetics can plan, track, correct, interact, become a marketplace portal member, and maintain a healthy lifestyle. In step 406, process 400 can use the application to manage/control patient obesity. In step 408, process 400 can patients use the application provides the much-needed guidance and tools for behavior modification, and manage anxiety and depression.

Process 400 showcases how patients (ad hoc or subscribers) can retain ownership of their digital health identity with the personalized EHR capability. Traditionally, this is done in conjunction with and with the permission of an intermediary healthcare institution—and the result is fragmentation of data, loss of control, silos of control, loss of data, and duplication of assessments and care. Ownership of their own record is in fact the next step to drive data ownership, control and responsibility of health awareness back to the individual or their designate. With this capability, patients can not only create their digital health identity but will have the ability for them to enter data from their own activity/wearables, while also allowing healthcare providers, known and ad hoc/unknown, to push patient-specific treatment information on to the patient profile using the blockchain.

In one example, process 400 can use a blockchain to create a patient identifier address and an associated public-private key pair for approving/signing transactions. Just as patients obtain identifiers, the provider (e.g. a hospital, insurance company, etc.) can create an identifier address and an associated public-private key pair. Permissions can then be associated with these identities typically granted by other entities on the blockchain. As an example, if the patient grants permission to a provider to access their current digital health record, only then does the provider get permission to access the profile of the patient and to use the information accordingly. To continue this example, the hospital can use the patient's public key to encrypt a medical record/summary message so that only the patient (e.g. with their private key) can decrypt and read said medical record/summary message. Such portable EHR must also allow existing identifiers on the blockchain (e.g. patient, a provider, etc.) to recover a lost private key.

When a patient uses a care provider for treatment/medications, the patient can use a private key to make such a treatment request. This request can also reside on the blockchain. In this way, the hospital will use the corresponding public key and verify that the treatment request message was created by the owner of the private key (and no one else). In such a manner, patients can securely share their identity, and engagement/treatment requests with a hospital. Using this embodiment or a similar technique, the hospital can send the summary care information securely/encrypted to the patient for aggregation onto their personalized longitudinal health record.

Process 400 can provide the ability to have an EHR that is updated in near-real time from providers, asynchronous updates from federal or state health agencies, and data streams from wearables—and becomes the essence of a portable EHR. This will be used in those markets where government agencies need to ensure that specific or emergency medical interventions are performed on tagged personal EHR (virtually impossible in today's EHRs).

Process 400 can be used to deliver care across a vast and distributed population. This can be the case with the restriction of capital constrained IT/system environments worldwide where there is a need to have an intermediary-less, real-time, immutable personal health record that travels with the patient. With permitted-access to the blockchain and most importantly, various healthcare organizations can access this personal patient data and/or population health dashboards to view key health metrics or to perform relevant interventions. This data can be used across language domains, so the personal health record has the ability to deliver the semantics of the patient data in a selected language on an as needed basis.

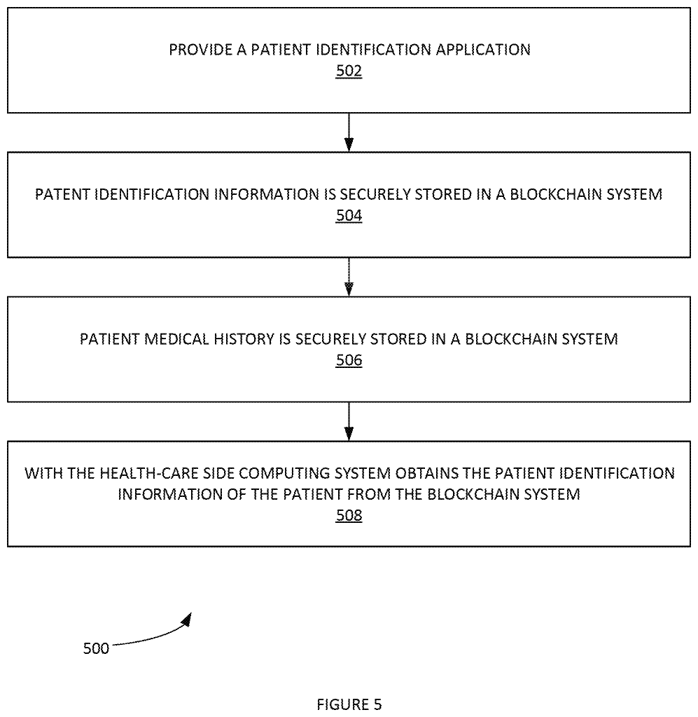

FIG. 5 illustrates an example process 500 for implementing a patient identification application to access patient information in a blockchain system, according to some embodiments. In step 502, process 500 can provide a patient identification application. The application can be provided to patient-side computing systems (e.g. mobile devices) and health-care side computing systems. In step 504, the patient identification information can be securely stored in a blockchain system (e.g. as a patient longitudinal health profile/record(s)). Example, patient identification information can be, inter alia: thumb impression, mobile device with RFID chips, other patient identifiers via patient's mobile device, care taker identifiers, iris scans, voice authentication, password/usernames combinations, etc. Other patent authentication information (e.g. passwords, usernames, biometric data, etc.) can be stored with the patient identification information as well. In step 506, the patient medical history can be securely stored in a blockchain system. In step 508, with the health-care side computing system can obtain the patient identification information of the patient from the blockchain system.

Process 500 can leverage blockchains to manage patient health issues. Process 500 can maintain an open marketplace for consumer demand. The decentralized of the blockchain system can empower patients.

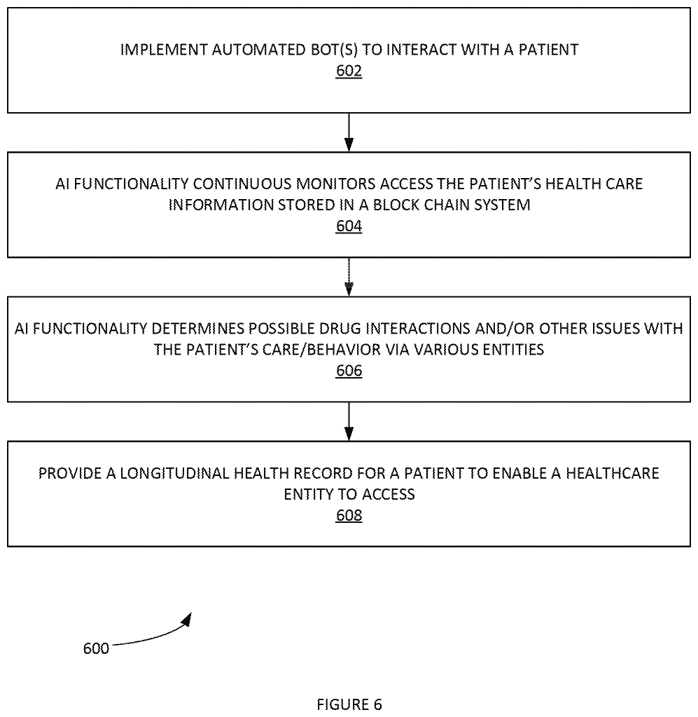

FIG. 6 illustrates an example process 600 for implementing an AI functionality to continuous monitor and advise patients, according to some embodiments. In step 602, process 600 can implement an automated bot(s) to interact with a patient. Process 600 can interact with patients via a patient-side application. In step 604, the AI functionality can continuous monitor access the patient's health care information stored in a block chain system. This information can be accessed from disparate locations (e.g. different medical offices, insurance entities, retail pharmacy entities, physical therapy entities, gym entities, etc.). In step 606, the AI functionality can determine possible drug interactions and/or other issues with the patient's care/behavior via various entities. In this way, process 600 can provide an eagles view of every provider to manage medication for the patient. In step 608, process 600 can provide a longitudinal health record for a patient to enable a healthcare entity to access. The patient can manage health with an AI guiding their actions: diet, medications, doctor visits, etc. In step 610, process 100 can implement tokenization of patient information and reward good behavior. As noted, via process 600 can share/scan their longitudinal health record with others and control access (e.g. later lock out a provider, etc.).

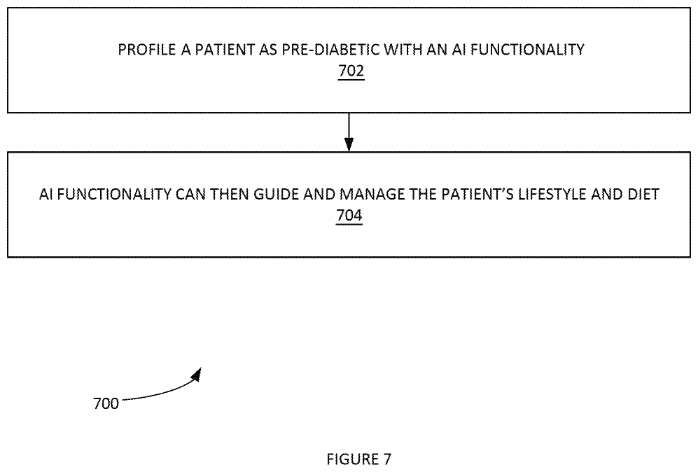

FIG. 7 illustrates an example process 700 for managing diabetes with a blockchain, according to some embodiments. In step 702, process 700 can profile a patient as pre-diabetic with an AI functionality. In step 704, the AI functionality can then guide and manage the patient's lifestyle and diet (e.g. via a mobile device application, etc.). In way, process 700 can provide day to day management skill sets so the patient doesn't have to depend on or wait for health care provider to provide guidance and/or feedback. Process 700 can provide interventions with the patient's diet and/or exercise routines in real-time. It is noted that process 700 can be modified for childhood obesity management in lieu of diabetes as well. Process 700 can provide food choices with ethnic preferences and monitor childhood exercise/play durations and behaviors via wearable devices. A wearable device can be an activity trackers that is a wireless-enabled wearable technology devices that measures data such as: the number of steps walked, heart rate, quality of sleep, steps climbed, and other personal metrics involved in fitness. This information can be stored in the patient information in the blockchain and/or included in the patient's blockchain-based longitudinal health record(s).

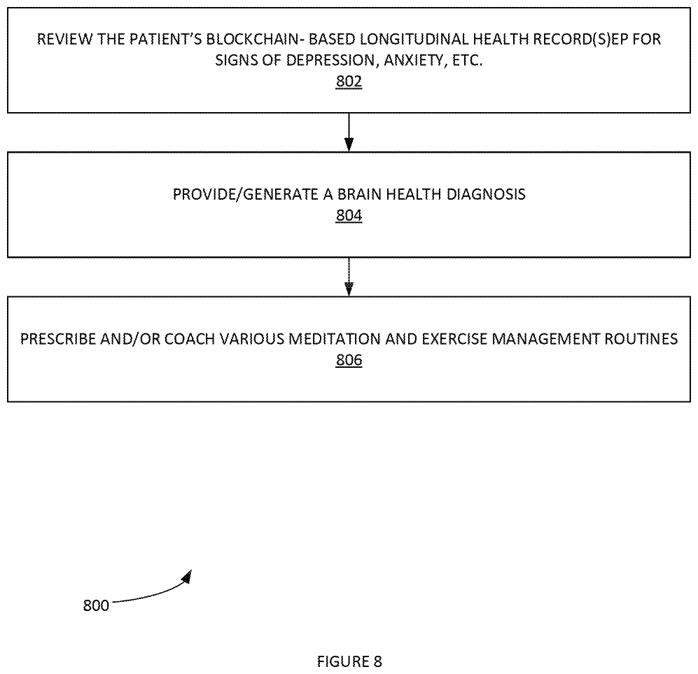

FIG. 8 illustrates an example process 800 for monitoring a patient's mental state with an AI functionality, according to some embodiments. In step 802, process 800 can review the patient's blockchain-based longitudinal health record(s)ep for signs of depression, anxiety, etc. In step 804, process 800 can provide/generate a brain health diagnosis. This can be done with integrative information and AI analysis. In step 806, process 800 can prescribe and/or coach (e.g. via a chat bot, etc.) various meditation and exercise management routines. Process 800 can supervise these routines and later patient behavior for modifications of patient mental-health issues. It is noted that at any time the patient can also upload mental-health information based on patient state to process 800. Process 800 can store this information in the patient's blockchain-based longitudinal health record(s).

Example Machine Learning Implementations

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning. Random forests (RF) (e.g. random decision forests) are an ensemble learning method for classification, regression and other tasks, that operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, that is a set of examples used to fit the parameters (e.g. weights of connections between neurons in artificial neural networks) of the model. The model (e.g. a neural net or a naive Bayes classifier) is trained on the training dataset using a supervised learning method (e.g. gradient descent or stochastic gradient descent). In practice, the training dataset often consist of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in a neural network). Validation datasets can be used for regularization by early stopping: stop training when the error on the validation dataset increases, as this is a sign of overfitting to the training dataset. This procedure is complicated in practice by the fact that the validation dataset's error may fluctuate during training, producing multiple local minima. This complication has led to the creation of many ad-hoc rules for deciding when overfitting has truly begun. Finally, the test dataset is a dataset used to provide an unbiased evaluation of a final model fit on the training dataset. If the data in the test dataset has never been used in training (for example in cross-validation), the test dataset is also called a holdout dataset.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed as new and desired to be protected by Letters Patent of the United States is:

1. A computerized method for implementing a patient identification application to access patient information in a blockchain system comprising:
providing a patient identification application, wherein the patient identification application is provided and implemented in a patient-side computing systems and health-care side computing systems;
securely storing a patient identification information in a blockchain system;
securely storing a patient medical history in the blockchain system; and
with a health-care side computing system, obtaining the patient identification information of the patient from the blockchain system.

2. The computerized method of claim 1, wherein the patient-side computing systems comprises a patient mobile device.

3. The computerized method of claim 2, wherein the patient identification information in the blockchain system comprises a patient longitudinal health record.

4. The computerized method of claim 3, wherein the patient identification information comprises a set of patient identifiers via the patient mobile device, a caretaker identifiers, an iris scan identifier of the patient, a voice authentication of the patient, and a password/usernames combination of the of the patient or the caretaker.

5. The computerized method of claim 3 further comprising:
using an artificial intelligence (AI) functionality to AI functionality to continuous monitor the patient identification information and the patient medical history; and
with the AI functionality generate a medical advice media to the patient based on the patient identification information and the patient medical history.

6. The computerized method of claim 5 further comprising:
implementing an automated chat bot to interact with the patient, wherein the automated bot interacts with the patient information and with patients medical history in the block chain system via a patient-side application.

7. The computerized method of claim 6,
wherein the AI functionality continuously monitors access the patient information and with patients medical history stored in the block chain system.

8. The computerized method of claim 7, wherein the AI functionality determines a set of drug interactions from the patient information and with patients medical history stored in the block chain.

9. The computerized method of claim 8 further comprising:
generating a longitudinal health record from the patient information and with patients medical history; and
storing the longitudinal health record in the block chain.

10. The computerized method of claim 9 further comprising:
providing access to the longitudinal health record for the patient to a specified healthcare entity to access.

11. The computerized method of claim 10 further comprising:
implementing a tokenization of the patient information.

12. The computerized method of claim 11 further comprising:
monitoring a patient's mental state with the AI functionality.

13. The computerized method of claim 12 further comprising:
with the AI functionality, reviewing the patient's blockchain-based longitudinal health record(s)ep for signs of the patient's mental state.

14. The computerized method of claim 13, wherein the patient's mental state comprises depression and anxiety.

15. The computerized method of claim 13, wherein based on the patient's mental state, the AI functionality generates a proposed patient mental state and communicates via the automated chatbot a meditation routine and an exercise management routine for the patient.
